(19) United States
(12) Patent Application Publication
(10) Pub. No.: US 2019/0038873 A1
(43) Pub. Date: Feb. 7, 2019

(54) STEERING ASSEMBLY FOR INTRAVASCULAR CATHETER SYSTEM

(71) Applicant: CRYTERION MEDICAL, INC., Carlsbad, CA (US)

(72) Inventors: Eric A. Schultheis, San Clemente, CA (US); Philip J. Simpson, Escondido, CA (US); Adam M. Ariely, Encinitas, CA (US); David G. Matsuura, Solana Beach, CA (US)

(21) Appl. No.: 16/044,914

(22) Filed: Jul. 25, 2018

Related U.S. Application Data
(60) Provisional application No. 62/560,464, filed on Sep. 19, 2017.
(60) Provisional application No. 62/541,586, filed on Aug. 4, 2017.

(51) Int. Cl. A61M 25/01

(52) U.S. Cl. CPC A61M 25/0147; A61M 2025/015; A61M 25/0136

(57) ABSTRACT

A steering assembly for an intravascular catheter system includes first and second pull wires, a steering knob, and first and second movers. The intravascular catheter system includes a handle assembly having a longitudinal axis and a balloon catheter having a catheter shaft with a distal end. The first and second pull wires extend between the handle assembly and the distal end of the catheter shaft. The first mover includes a knob engager that engages an internal thread of the steering knob so that rotation of the steering knob about the longitudinal axis moves the first mover in a first direction. The second mover is coupled to the first mover so that the second mover moves in a second direction. Movement of the first and second movers moves the first and second pull wires to articulate a portion of the balloon catheter near the distal end of the catheter shaft.

### RELATED APPLICATIONS

This application claims priority on U.S. Provisional Application Ser. No. 62/541,586 filed on Aug. 4, 2017 and entitled “CATHETER STEERING DEVICE FOR AN INTRAVASCULAR CATHETER SYSTEM” and U.S. Provisional Application Ser. No. 62/560,464 filed on Sep. 19, 2017 and entitled “CATHETER STEERING ASSEMBLY FOR AN INTRAVASCULAR CATHETER SYSTEM”. As far as permitted, the contents of U.S. Provisional Application Ser. Nos. 62/541,586 and 62/560,464 are incorporated in their entirety herein by reference.

### BACKGROUND

Cardiac arrhythmias involve an abnormality in the electrical conduction of the heart and are a leading cause of stroke, heart disease, and sudden cardiac death. Treatment options for patients with arrhythmias include medications, implantable devices, and catheter ablation of cardiac tissue.

Catheter ablation involves delivering ablative energy to tissue inside the heart to block aberrant electrical activity from depolarizing heart muscle cells out of synchrony with the heart's normal conduction pattern. The procedure is performed by positioning a portion of an energy delivery catheter adjacent to diseased or targeted tissue in the heart. The energy delivery component of the system is typically at or near a most distal (farthest from the operator) portion of the catheter, and often at a tip of the device. Various forms of energy are used to ablate diseased heart tissue. These can include radio frequency (RF), ultrasound and laser energy, to name a few. One form of energy that is used to ablate diseased heart tissue includes cryogenics (also referred to herein as “cryoablation”). During a cryoablation procedure, with the aid of a guidewire, the distal tip of the catheter is positioned adjacent to diseased or targeted tissue, at which time the cryogenic energy can be delivered to create tissue necrosis, rendering the ablated tissue incapable of conducting electrical signals.

Atrial fibrillation is one of the most common arrhythmias treated using cryoablation. In the earliest stages of the disease, paroxysmal atrial fibrillation, the treatment strategy involves isolating the pulmonary veins from the left atrial chamber, a procedure that removes unusual electrical conductivity in the pulmonary vein. Recently, the use of techniques known as “balloon cryotherapy” catheter procedures to treat atrial fibrillation have increased. In part, this stems from ease of use, shorter procedure times and improved patient outcomes. During the balloon cryotherapy procedure, a refrigerant or cryogenic fluid (such as nitrous oxide, or any other suitable fluid) is delivered under pressure to an interior of one or more inflatable balloons which are positioned adjacent to or against the targeted cardiac tissue. Using this method, the extremely frigid cryogenic fluid causes necrosis of the targeted cardiac tissue, thereby rendering the ablated tissue incapable of conducting unwanted electrical signals.

During cryoablation procedures, the distal end of the catheter is designed to reach tissue within the patient's heart. In order to reach various locations within the heart, the procedure requires that the catheter be carefully steered or navigated through the patient's body, particularly the patient's vascular path. Navigation of the catheter is generally performed with the use of pull wire(s) that typically extend from within a handle assembly and run distally through the wall of a catheter sheath and/or catheter shaft. Specifically, manipulating the pull wire(s) causes a distal end of the catheter to articulate, allowing the catheter to be steered, navigated and/or ultimately positioned advantageously in a region of interest for the cryoablation procedure. In other words, the articulation of the distal end of the catheter is generally realized by the actuation, i.e., push or pull motion, of the pull wire(s) within the handle assembly. The wide ranging forces used on the pull wire(s) to articulate the distal end of the catheter can often result in stresses or forces that may cause kinks and/or twisting of the pull wire(s). Due to these additional forces, it is not uncommon for the pull wire(s) to fatigue and/or breakdown. Any kinks and/or twisting of the pull wire(s) during the cryoablation procedure would not only interrupt the procedure, but could also be injurious to the patient.

Additionally, the handle assembly generally includes a steering knob that controls complex configurations of several working components within the handle assembly in order to achieve the actuation of the pull wire(s). Due to the complex configurations, the steering knob generally requires excessive force and/or rotations be applied. Such configurations also include sequentially nested and threaded components which make manufacturing inefficient and often cause deficient actuation due to imprecise component interactions. Further, the configurations often require the handle assembly design to be relatively elongated or bulky in order to accommodate the several working components.

### SUMMARY

The present invention is directed toward a steering assembly for an intravascular catheter system (sometimes referred to herein as “catheter system). In various embodiments, the catheter system can include a handle assembly and a balloon catheter. The handle assembly can have a longitudinal axis. The balloon catheter can have a catheter shaft that extends from the handle assembly. Additionally, the catheter shaft can have a distal end.

In various embodiments, the steering assembly includes a first pull wire, a steering knob and a first mover. In one embodiment, the first pull wire can extend between the handle assembly and the distal end of the catheter shaft. In certain embodiments, the steering knob can have an internal thread.

The steering knob can be rotatable about the longitudinal axis. In one embodiment, the steering knob can be coupled to the handle assembly. In another embodiment, the steering knob can encircle at least a portion of the handle assembly. Additionally, in certain embodiments, the steering knob can rotate relative to the first mover.

In some embodiments, the first mover is connected to the first pull wire. Additionally, the first mover can include a knob engager that engages the internal thread of the steering knob. Accordingly, in various embodiments, as the steering knob is rotated, the steering knob can move the first mover in a direction that is substantially parallel to the longitudinal axis. In an alternative embodiment, the first mover can move along the longitudinal axis. Furthermore, such movement of the first mover can move the first pull wire which can cause a portion of the balloon catheter at or near the distal end of the catheter shaft to articulate. In an alternative embodiment, such movement of the first mover can move the first pull wire to articulate a portion of the balloon catheter at or near a distal end of a catheter sheath.

The present invention is also directed toward a steering assembly for an intravascular catheter system. In certain embodiments, the catheter system can include a handle assembly and a balloon catheter. The handle assembly can have a longitudinal axis. The balloon catheter can have a catheter shaft that extends from the handle assembly. Additionally, the catheter shaft can have a distal end.

In certain embodiments, the steering assembly includes a first pull wire, a second pull wire, a steering knob, a first mover and a second mover. In one embodiment, the first pull wire can extend between the handle assembly and the distal end of the catheter shaft. In another embodiment, the second pull wire can also extend between the handle assembly and the distal end of the catheter shaft.

In certain embodiments, the steering knob can have an internal thread. The steering knob can be rotatable about the longitudinal axis. In one embodiment, the steering knob can be coupled to the handle assembly. In another embodiment, the steering knob can encircle at least a portion of the handle assembly. Additionally, in certain embodiments, the steering knob can rotate relative to the first mover and the second mover.

In various embodiments, the first mover is connected to the first pull wire. Additionally, the first mover can include a knob engager that engages the internal thread of the steering knob. Accordingly, in various embodiments, as the steering knob is rotated, the steering knob can move the first mover in a first direction that is substantially parallel to the longitudinal axis. In an alternative embodiment, the first mover can move along the longitudinal axis.

In certain embodiments, the second mover is connected to the second pull wire. Additionally, the second mover can be coupled to the first mover. Accordingly, in various embodiments, as the first mover moves in the first direction, the second mover can move in a second direction that is substantially parallel to the longitudinal axis. In an alternative embodiment, the second mover can move along the longitudinal axis. Furthermore, such movement of the first mover and the second mover can move the first pull wire and the second pull wire, which can cause a portion of the balloon catheter at or near the distal end of the catheter shaft to articulate. Alternatively, such movement of the first mover and the second mover can move the first pull wire and the second pull wire, which can cause a portion of the balloon catheter at or near a distal end of the catheter sheath to articulate.

In some embodiments, the first mover and the second mover can be substantially parallel to each other. For example, in one embodiment, the first mover can be positioned on top of the second mover.

In other embodiments, the steering assembly can further include a first rack and a second rack. In such embodiments, the first rack can be connected to the first mover and the second rack can be connected to the second mover. In various embodiments, the steering assembly can also include a pinion. The pinion can include a helical design or a spur design, as non-exclusive examples. In certain embodiments, the pinion can couple the first mover and the second mover to each other. Further, the pinion can rotate relative to the first mover and the second mover. In some embodiments, the pinion can engage the first rack and the second rack.

Additionally, in some applications, the present invention is directed toward a steering assembly for a catheter system. In certain embodiments, the catheter system can include a handle assembly and a balloon catheter. The handle assembly can have a longitudinal axis. The balloon catheter can have a catheter shaft that extends from the handle assembly. Additionally, the catheter shaft can have a distal end

In some embodiments, the steering assembly includes a first pull wire, a second pull wire, a steering knob, a first mover, a second mover and a pinion. In one embodiment, the first pull wire can extend between the handle assembly and the distal end of the catheter shaft. In another embodiment, the second pull wire can also extend between the handle assembly and the distal end of the catheter shaft.

In various embodiments, the steering knob can have an internal thread. The steering knob can be rotatable about the longitudinal axis.

In various embodiments, the first mover is connected to the first pull wire. The first mover can also include a first rack. Additionally, the first mover can include a knob engager that engages the internal thread of the steering knob. Accordingly, in various embodiments, as the steering knob is rotated, the steering knob can move the first mover in a first direction that is substantially parallel to the longitudinal axis.

In certain embodiments, the second mover is connected to the second pull wire. The second mover can include a second rack.

In some embodiments, the pinion can engage the first rack and the second rack. Accordingly, in various embodiments, as the first mover moves in the first direction, the second mover can move in a second direction that is substantially parallel to the longitudinal axis. Furthermore, such movement of the first mover and the second mover can move the first pull wire and the second pull wire, which can cause a portion of the balloon catheter at or near the distal end of the catheter shaft to articulate.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic view of a patient and one embodiment of an intravascular catheter system having features of the present invention;

FIG. 2 is a simplified side view of an embodiment of a portion of the intravascular catheter system, including one embodiment of a portion of a catheter steering assembly;

FIG. 3A is a perspective view of an embodiment of a handle assembly and another embodiment of the catheter steering assembly;

FIG. 3B is a cross-sectional view of a portion of the embodiment of the handle assembly and a portion of the embodiment of the steering assembly taken on line 3B-3B in FIG. 3A;

FIG. 4A is a perspective view of another embodiment of the handle assembly and still another embodiment of the catheter steering assembly;

FIG. 4B is a cross-sectional view of a portion of the embodiment of the handle assembly and a portion of the embodiment of the steering assembly taken on line 4B-4B in FIG. 4A; and

FIG. 5 is a perspective view of a portion of yet another embodiment of the catheter steering assembly.

### DESCRIPTION

Embodiments of the present invention are described herein in the context of a catheter steering assembly (also sometimes referred to herein as a “steering assembly”) for an intravascular catheter system. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Although the disclosure provided herein focuses mainly on cryogenics, it is understood that various other forms of energy can be used to ablate diseased heart tissue. These can include radio frequency (RF), ultrasound, pulsed DC electric fields and laser energy, as non-exclusive examples. The present invention is intended to be effective with any or all of these and other forms of energy.

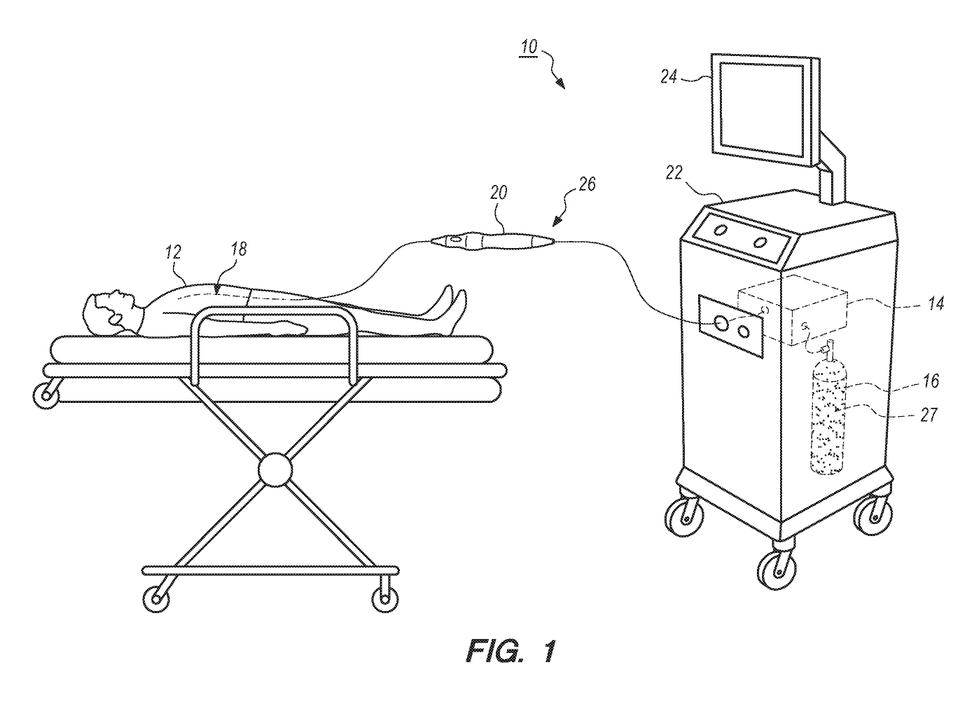

FIG. 1 is a schematic view of one embodiment of an intravascular catheter system 10 (also sometimes referred to as a “catheter system”) for use with a patient 12, which can be a human being or an animal. Although the catheter system 10 is specifically described herein with respect to the intravascular catheter system, it is understood and appreciated that other types of catheter systems and/or ablation systems can equally benefit by the teachings provided herein. For example, in certain non-exclusive alternative embodiments, the present invention can be equally applicable for use with any suitable types of ablation systems and/or any suitable types of catheter systems. Thus, the specific reference herein to use as part of the intravascular catheter system is not intended to be limiting in any manner.

The design of the catheter system 10 can be varied. In certain embodiments, such as the embodiment illustrated in FIG. 1, the catheter system 10 can include one or more of a control system 14, a fluid source 16 (e.g., one or more fluid containers), a balloon catheter 18, a handle assembly 20, a control console 22, a graphical display 24 (also sometimes referred to as a graphical user interface or “GUI”) and a steering assembly 26. It is understood that although FIG. 1 illustrates the structures of the catheter system 10 in a particular position, sequence and/or order, these structures can be located in any suitably different position, sequence and/or order than that illustrated in FIG. 1. It is also understood that the catheter system 10 can include fewer or additional structures than those specifically illustrated and described herein.

In various embodiments, the control system 14 is configured to monitor and control the various processes of a cryoablation procedure. More specifically, the control system 14 can monitor and control release and/or retrieval of a cryogenic fluid 27 to and/or from the balloon catheter 18. The control system 14 can also control various structures that are responsible for maintaining or adjusting a flow rate and/or a pressure of the cryogenic fluid 27 that is released to the balloon catheter 18 during the cryoablation procedure. In such embodiments, the catheter system 10 delivers ablative energy in the form of cryogenic fluid 27 to cardiac tissue of the patient 12 to create tissue necrosis, rendering the ablated tissue incapable of conducting electrical signals. Additionally, in various embodiments, the control system 14 can control activation and/or deactivation of one or more other processes of the balloon catheter 18. Further, or in the alternative, the control system 14 can receive electrical signals, data and/or other information (also sometimes referred to as “sensor output”) from various structures within the catheter system 10. In various embodiments, the control system 14 and/or the GUI 24 can be electrically connected and/or coupled. In some embodiments, the control system 14 can receive, monitor, assimilate and/or integrate any sensor output and/or any other data or information received from any structure within the catheter system 10 in order to control the operation of the balloon catheter 18. Still further, or in the alternative, the control system 14 can control positioning of portions of the balloon catheter 18 within a circulatory system (not shown) (also sometimes referred to herein as the “body”) of the patient 12, and/or can control any other suitable functions of the balloon catheter 18.

The fluid source 16 (also sometimes referred to as “fluid container 16”) can include one or more fluid container(s) 16. It is understood that while one fluid container 16 is illustrated in FIG. 1, any suitable number of fluid containers 16 may be used. The fluid container(s) 16 can be of any suitable size, shape and/or design. The fluid container(s) 16 contains the cryogenic fluid 27, which is delivered to the balloon catheter 18 with or without input from the control system 14 during the cryoablation procedure. Once the cryoablation procedure has initiated, the cryogenic fluid 27 can be injected or delivered and the resulting gas, after a phase change, can be retrieved from the balloon catheter 18, and can either be vented or otherwise discarded as exhaust (not shown). More specifically, the cryogenic fluid 27 delivered to and/or removed from the balloon catheter 18 can include a flow rate that varies. Additionally, the type of cryogenic fluid 27 that is used during the cryoablation procedure can vary. In one non-exclusive embodiment, the cryogenic fluid 27 can include liquid nitrous oxide. In another non-exclusive embodiment, the cryogenic fluid 27 can include liquid nitrogen. However, any other suitable cryogenic fluid 27 can be used.

The design of the balloon catheter 18 can be varied to suit the design requirements of the catheter system 10. As shown, the balloon catheter 18 is inserted into the body of the patient 12 during the cryoablation procedure. In one embodiment, the balloon catheter 18 can be positioned within the body of the patient 12 using the control system 14. Stated in another manner, the control system 14 can control positioning of the balloon catheter 18 within the body of the patient 12. Alternatively, the balloon catheter 18 can be manually positioned within the body of the patient 12 by a qualified healthcare professional (also referred to herein as an “operator”). As used herein, healthcare professional and/or operator can include a physician, a physician's assistant, a nurse and/or any other suitable person or individual. In certain embodiments, the balloon catheter 18 is positioned within the body of the patient 12 utilizing at least a portion of the sensor output that is received from the balloon catheter 18. For example, in various embodiments, the sensor output is received by the control system 14, which can then provide the operator with information regarding the positioning of the balloon catheter 18. Based at least partially on the sensor output feedback received by the control system 14, the operator can adjust the positioning of the balloon catheter 18 within the body of the patient 12 to ensure that the balloon catheter 18 is properly positioned relative to targeted cardiac tissue. While specific reference is made herein to the balloon catheter 18, as noted above, it is understood that any suitable type of medical device and/or catheter may be used.

The handle assembly 20 is handled and used by the operator to operate, position and control the balloon catheter 18. The design and specific features of the handle assembly 20 can vary to suit the design requirements of the catheter system 10. In the embodiment illustrated in FIG. 1, the handle assembly 20 is separate from, but in electrical and/or fluid communication with the control system 14, the fluid container 16 and the GUI 24. In some embodiments, the handle assembly 20 can integrate and/or include at least a portion of the control system 14 and/or steering assembly 26 within an interior of the handle assembly 20. In one embodiment, an operator can steer and/or navigate the balloon catheter 18 by utilizing the handle assembly 20 and/or the steering assembly 26. It is understood that the handle assembly 20 can include fewer or additional components than those specifically illustrated and described herein.

In the embodiment illustrated in FIG. 1, the control console 22 includes at least a portion of the control system 14, the fluid container 16 and/or the GUI 24. However, in alternative embodiments, the control console 22 can contain additional structures not shown or described herein. Still alternatively, the control console 22 may not include various structures that are illustrated within the control console 22 in FIG. 1. For example, in certain non-exclusive alternative embodiments, the control console 22 does not include the GUI 24.

In various embodiments, the GUI 24 is electrically connected to the control system 14. Additionally, the GUI 24 provides the operator of the catheter system 10 with information that can be used before, during and after the cryoablation procedure. For example, the GUI 24 can provide the operator with information based on the sensor output, and any other relevant information that can be used before, during and after the cryoablation procedure. The specifics of the GUI 24 can vary depending upon the design requirements of the catheter system 10, or the specific needs, specifications and/or desires of the operator.

In one embodiment, the GUI 24 can provide static visual data and/or information to the operator. In addition, or in the alternative, the GUI 24 can provide dynamic visual data and/or information to the operator, such as video data or any other data that changes over time, e.g., during the cryoablation procedure. Further, in various embodiments, the GUI 24 can include one or more colors, different sizes, varying brightness, etc., that may act as alerts to the operator. Additionally, or in the alternative, the GUI 24 can provide audio data or information to the operator.

As an overview, and as provided in greater detail herein, the steering assembly 26 can be configured to articulate, allowing the balloon catheter 18 to be steered, navigated and/or ultimately positioned within the body of the patient 12 during the cryoablation procedure. As used herein, it is understood that the term “articulate” can include bend, turn, deflect, curve, or any other non-linear movement. In the embodiment illustrated in FIG. 1, at least a portion of the steering assembly 26 is integrated with and/or positioned on or within the handle assembly 20 and/or the balloon catheter 18. The steering assembly 26 can be positioned at any suitable location on or within the handle assembly 20 and/or the balloon catheter 18. Additionally, and/or in the alternative, at least a portion of the steering assembly 26 can be integrated with and/or positioned on or within any other suitable structure of the catheter system 10.

The specific components and operations of the steering assembly 26 will be described in greater detail herein in relation to the embodiments illustrated in the drawings. It is appreciated that the drawings included herewith may not necessarily be drawn to scale. Additionally, it is further appreciated that the drawings may not precisely represent the structures or components of the catheter system 10 and/or steering assembly 26, but are included for purposes of clarity in demonstrating certain features and limitations of the catheter system 10 and/or steering assembly 26.

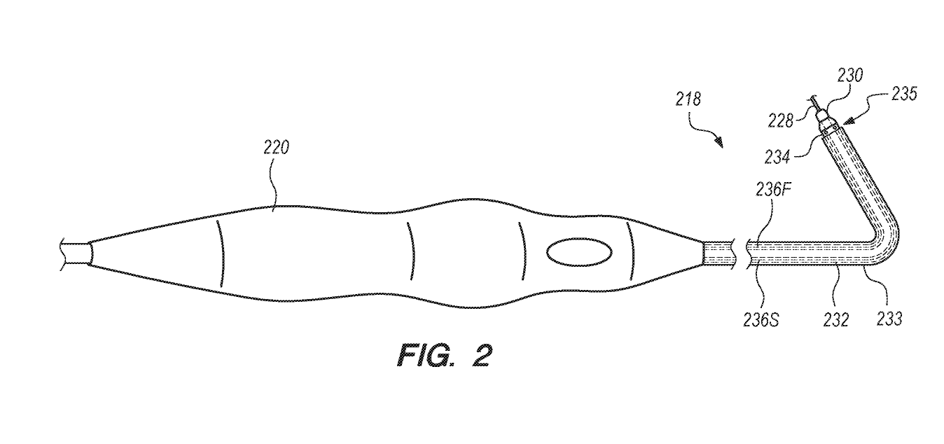

FIG. 2 is a simplified side view of an embodiment of a portion of the catheter system 210, including an embodiment of a portion of the steering assembly 226. In the embodiment illustrated in FIG. 2, the catheter system 210 includes the balloon catheter 218, the handle assembly 220 and a portion of the steering assembly 226.

In the embodiment illustrated in FIG. 2, at least a portion of the balloon catheter 218 can be inserted into the body of the patient 12 (illustrated in FIG. 1) during the cryoablation procedure. In this embodiment, the balloon catheter 218 can include one or more of a guidewire 228, a guidewire lumen 230, a catheter shaft 232, a catheter sheath 233 and a steering anchor 234. It is understood that the balloon catheter 218 can include other structures as well that are not shown and/or described relative to FIG. 2.

The guidewire 228 and/or guidewire lumen 230 are inserted into the body of the patient 12, and the catheter shaft 232 is moved along the guidewire 228 and/or guidewire lumen 230 to near an ostium (not shown) of a pulmonary vein (not shown) of the patient 12. Accordingly, the catheter shaft 232 can include a distal end 235 that extends to and/or can be at or near the ostium of the pulmonary vein of the patient 12. The catheter sheath 233 can also be moved along catheter shaft 232 to near the ostium of the pulmonary vein of the patient 12. In various embodiments, the guidewire 228, guidewire lumen 230, catheter shaft 232 and/or catheter sheath 233 can extend between the handle assembly 220 to at or near the ostium of the pulmonary vein of the patient 12. As referred to herein, it is understood that “at or near the distal end 235 of the catheter shaft 232” can also include at or near the distal end 235 of the catheter sheath 233, or at or near the distal end 235 of the guidewire lumen 230. As such, the distal end 235 of the guidewire lumen 230, the catheter shaft 232 and/or the catheter sheath 233 can include the portion of the balloon catheter 218 that can extend to and/or can be at or near the desired location within the body of the patient 12, e.g., at or near the ostium of the pulmonary vein.

The design and/or configuration of the steering anchor 234 can vary. In one non-exclusive embodiment, the steering anchor 234 can have a ring-shaped configuration. In alternative embodiments, the steering anchor 234 can include any other suitable configuration. In certain embodiments, the steering anchor 234 can be coupled, secured or connected to the balloon catheter 218, such as to a wall and/or an interior of the guidewire lumen 230, the catheter shaft 232 or the catheter sheath 233. The steering anchor 234 can be coupled, secured or connected to the wall and/or the interior of the guidewire lumen 230, the catheter shaft 232 or the catheter sheath 233 with the use of an adhesive or a thermal bonding technique, as non-exclusive examples. Alternatively, the steering anchor 234 may be coupled, secured or connected to the balloon catheter 218 in any other suitable manner which allows the operator to articulate the balloon catheter 218 in order to ultimately steer, navigate and/or advantageously position the balloon catheter 218 to a desired location, e.g., at or near the ostium of the pulmonary vein of the patient 12. Additionally, the steering anchor 234 may be made from any suitable material or materials, such as stainless steel or plastic, as non-exclusive examples.

The steering anchor 234 can be positioned anywhere along the length of the balloon catheter 218, including within the wall and/or the interior of the guidewire lumen 230, the catheter shaft 232 or the catheter sheath 233. For example, the steering anchor 234 can be positioned distally (away from) from the handle assembly 220 along a portion of the balloon catheter 218, such as at or near the distal end 235 of the catheter shaft 232. In the embodiment illustrated in FIG. 2, the steering anchor 234 is positioned within the catheter shaft 232, at or near the distal end 235 of the catheter shaft 232. Alternatively, the steering anchor 234 can be positioned within the catheter sheath 233, at or near the distal end 235 of the catheter sheath 233.

The steering assembly 226 can allow the balloon catheter 218 to be articulated in order to steer, navigate and/or advantageously position the balloon catheter 218 during the cryoablation procedure. More specifically, the steering assembly 226 can be configured to articulate a portion of the balloon catheter 218 at or near the distal end 235 of the catheter shaft 232. The design of the steering assembly 226 can vary. In the embodiment illustrated in FIG. 2, only a portion of the steering assembly 226, including a first pull wire 236F and a second pull wire 236S, is shown. As referred to herein, the first pull wire 236F and the second pull wire 236S may be used interchangeably. Additionally, while FIG. 2 illustrates the steering assembly 226 having pull wires 236F, 236S, it is understood that the steering assembly 226 may include any number of pull wires 236F, 236S. It is further understood that the steering assembly 226 can include additional components than those specifically illustrated and described herein.

In certain embodiments, the first pull wire 236F and the second pull wire 236S can extend generally between a location within the handle assembly 220 and the steering anchor 234. The first pull wire 236F and the second pull wire 236S may also extend generally between a location within the handle assembly 220 and the distal end 235 of the catheter shaft 232. The pull wires 236F, 236S, can be coupled, secured or connected to the handle assembly 220, which may allow the pull wires 236F, 236S, to be maneuvered or manipulated by the operator to articulate the guidewire lumen 230, catheter shaft 232 and/or catheter sheath 233, to ultimately position the balloon catheter 218 at or near the ostium of the pulmonary vein of the patient 12 during the cryoablation procedure. The pull wires 236F, 236S, can be coupled, secured or connected to the handle assembly 220 in any suitable manner. Additionally, the pull wires 236F, 236S, can also be coupled, secured or connected to the steering anchor 234. The pull wires 236F, 236S, may be coupled, secured or connected to the steering anchor 234 via any suitable manner, including weld or solder joint, adhesive or bonding material, as non-exclusive examples.

In certain embodiments, the pull wires 236F, 236S, can have a circular cross-section. In alternative embodiments, the pull wires 236F, 236S, can have the cross-section of any other suitable design and/or shape. Further, the pull wires 236F, 236S, may be made from any suitable material or materials.

It is recognized that the simplified steering assembly 226 illustrated in FIG. 2 is for representative purposes only. The specific components and operation of the steering assembly 226 will be further described in greater detail herein.

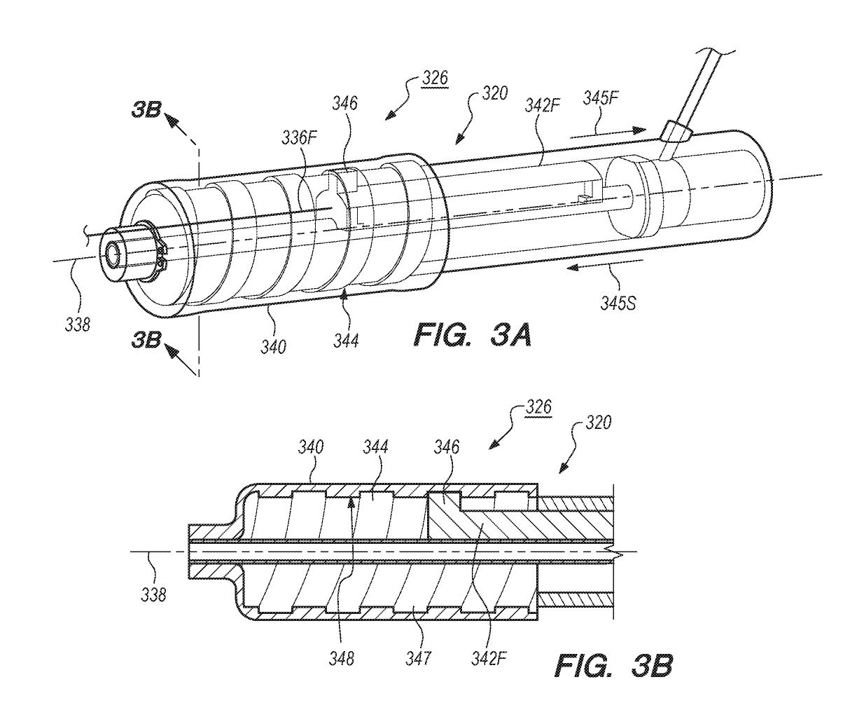

FIG. 3A is a perspective view of an embodiment of the handle assembly 320 and another embodiment of the steering assembly 326. In FIG. 3A, the handle assembly 320 is substantially cylindrical, having a longitudinal axis 338 (illustrated as a dashed line). As used herein, the term “substantially” is intended to allow for minor deviations. In alternative embodiments, the handle assembly 320 can include any other suitable design. Furthermore, in certain embodiments, such as the embodiment illustrated in FIG. 3A, at least a portion of the steering assembly 326 can be integrated and/or included with the handle assembly 320, which may include an interior of the handle assembly 320.

In the embodiment illustrated in FIG. 3A, the steering assembly 326 can include one or more of the first pull wire 336F, a steering knob 340 and a first mover 342F. It is understood that the steering assembly 326 can include fewer or additional components than those specifically illustrated and described herein.

In certain embodiments, the first pull wire 336F can extend between the handle assembly 320 and the distal end 235 of the catheter shaft 232 (illustrated in FIG. 2). Alternatively, the first pull wire 336F can extend between the handle assembly 320 and the steering anchor 234 (illustrated in FIG. 2). In one embodiment, the first pull wire 336F can extend between the handle assembly 320 and the distal end 235 of the catheter shaft 232 by being at least partially embedded within the wall (not shown) of the catheter shaft 232. In another embodiment, the first pull wire 336F can be located within the interior (not shown) of the catheter shaft 232. Alternatively, the first pull wire 336F can extend between the handle assembly 320 and the distal end 235 of the catheter shaft 232 by being at least partially embedded within the wall (not shown) and/or the interior (not shown) of the guidewire lumen 230. Additionally, and/or in the alternative, the first pull wire 336F can extend between the handle assembly 320 and the distal end 235 of the catheter shaft 232 via any suitable manner.

In the embodiment illustrated in FIG. 3A, the steering knob 340 can be manipulated by the operator to rotate about the longitudinal axis 338. The design of the steering knob 340 can vary. In FIG. 3A, the steering knob 340 is substantially cylindrical and is coupled to and/or at least partially encircles or surrounds a portion of the handle assembly 320. Accordingly, the steering knob 340 can also include a cylindrical hole or shaft that at least partially encircles or surrounds a portion of the handle assembly 320 or other components of the steering assembly 326. Both the steering knob 340 and the handle assembly 320 may include or share the same longitudinal axis 338. In other embodiments, the steering knob 340 can include any other suitable shape or design. Additionally and/or alternatively, the steering knob 340 can be integrated with other structures within the catheter system 10.

In certain embodiments, the steering knob 340 can include an internal thread 344. The internal thread 344 may positioned on an interior of the steering knob 340. In the embodiment illustrated in FIG. 3A, the internal thread 344 can include a right-handed thread or a left-handed thread. In alternative embodiments, the internal thread 344 can include any other threaded design. Furthermore, the width of the threaded design can vary.

The first mover 342F is configured to move along the longitudinal axis 338, i.e., in a first direction 345F and in a second direction 345S that is opposite the first direction 345F (sometimes collectively referred to herein as “direction”). In this embodiment, the first direction 345F is shown to be in a backward direction, while the second direction 345S is shown to be in a forward direction. The first direction 345F and the second direction 345S can be interchangeable so long as the first direction 345F is opposite the second direction 345S, and vice versa. In some embodiments, the first mover 342F can move in a direction that is substantially parallel to the longitudinal axis 338 in the first direction 345F and the second direction 345S.

The design of the first mover 342F can vary. In the embodiment illustrated in FIG. 3A, the first mover 342F is engaged with the steering knob 340, such that as the steering knob 340 is manipulated, i.e., rotated about the longitudinal axis 338 in a clockwise or counter-clockwise direction, the first mover 342F can move relative to the steering knob 334. In other words, the steering knob 340 can rotate relative to the first mover 342F. The first mover 342F can be engaged with the steering knob 334 via any suitable manner or method. For example, in FIG. 3A, the first mover 342F is engaged with the internal thread 344 of the steering knob 340 such that the internal thread 344 can act to push or move the first mover 342F in the first direction 345F and the second direction 345S. More specifically, the first mover 342F can include a knob engager 346 that engages or slots into the internal thread 344 on the interior of the steering knob 340. In this embodiment, the knob engager 346 is positioned on a top surface of the first mover 342F. Accordingly, as the steering knob 340 is rotated about the longitudinal axis 338, the rotational motion of the steering knob 340 and the internal thread 344 can cause simultaneous movement of the first mover 342F in the first direction 345F or the second direction 345S, thereby translating the rotational motion of the steering knob 340 into linear movement of the first mover 342F.

In some embodiments, at least a portion of the first mover 342F can be at least partially positioned within the interior of the steering knob 340 and/or within the interior of the handle assembly 320. In other embodiments, the first mover 342F can be positioned solely within the interior of the handle assembly 320.

In various embodiments, the first pull wire 336F can be coupled, secured or connected to the first mover 342F. While in the embodiment illustrated in FIG. 3A, the first pull wire 336F is coupled, secured or connected to the first mover 342F at or near an end that is adjacent to the steering knob 340, it is understood that the first pull wire 336F can be coupled, secured or connected to the first mover 342F at any other location or position. Additionally, the first pull wire 336F can be coupled, secured or connected to the first mover 342F in any suitable manner, i.e., weld or solder joint, adhesive or bonding material, as non-exclusive examples. In certain embodiments, as the first mover 342F moves, the first pull wire 336F is moved. For example, in FIG. 3A, when the steering knob 340 is manipulated, i.e., rotated about the longitudinal axis 338, the first mover 342F moves in the first direction 345F or the second direction 345S, which in turn causes movement of the first pull wire 336F. The movement of the first pull wire 336F functions to push or loosen, or pull or tighten the first pull wire 336F, which can cause articulation of a portion of the balloon catheter 218 (illustrated in FIG. 2). Stated another way, when the operator rotates the steering knob 340, a portion of the balloon catheter 218 at or near the distal end 235 of the catheter shaft 232 may then be articulated in the direction of the force being exerted by the first pull wire 336F.

FIG. 3B is a cross-sectional view of a portion of the embodiment of the handle assembly 320 and a portion of the embodiment of the steering assembly 326 taken on line 3B-3B in FIG. 3A. In this embodiment, both the steering knob 340 and the handle assembly 320 include or share the same longitudinal axis 338 (illustrated as a dashed line).

In the embodiment illustrated in FIG. 3B, the steering knob 340 includes a cylindrical cavity or shaft 347 that at least partially encircles or surrounds a portion of the handle assembly 320 and/or houses other components of the steering assembly 326. More specifically, the cylindrical cavity or shaft 347 at least partially houses the first drive member 342F, such that the first drive member 342F can move in the first direction 345F (illustrated in FIG. 3A) and the second direction 345S (illustrated in FIG. 3A), while within the cylindrical cavity or shaft 347 of the steering knob 340.

Additionally, in this embodiment, the steering knob 340 includes the internal thread 344. The internal thread 344 is positioned on an inner surface 348 of the steering knob 340, such that the knob engager 346 can engage the internal thread 344 of the steering knob 340. With this configuration, as the steering knob 340 is manipulated, i.e., rotated about the longitudinal axis 338 in a clockwise or counter-clockwise direction, the first mover 342F can move in the first direction 345F or the second direction 345S.

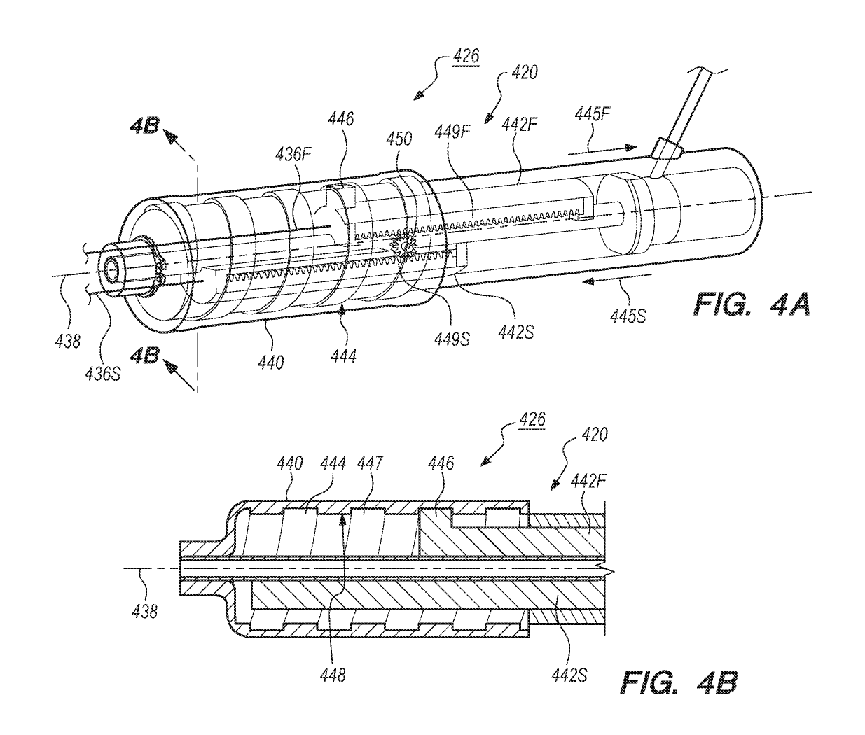

FIG. 4A is a perspective view of another embodiment of the handle assembly 420 and still another embodiment of the steering assembly 426. In the embodiment illustrated in FIG. 4A, the steering assembly 426 includes the first pull wire 436F, the second pull wire 436S, the steering knob 440, the first mover 442F and a second mover 442S. As referred to herein, the first mover 442F and the second mover 442S may be used interchangeably. Further, while FIG. 4A illustrates the steering assembly 426 having movers 442F, 442S, it is understood that the steering assembly 426 may include any number of movers 442F, 442S.

The design of the first mover 442F and the second mover 442S can vary. In the embodiment illustrated in FIG. 4A, a first rack 449F is secured or connected to the first mover 442F. In certain embodiments, the first rack 449F can include a linear gear bar design. Alternatively, the first rack 449F can include any other suitable design. Additionally, a second rack 449S is secured or connected to the second mover 442S. The second rack 449S can also include a linear gear bar design or any other suitable design. The racks 449F, 449S, can be secured or connected to the movers 442F, 442S, via any suitable manner or method and at any suitable location. It is understood that “the first rack 449F” and “the second rack 449S” can be used interchangeably.

The positioning of the movers 442F, 442S, can also vary. For example, in some embodiments, the first mover 442F and the second mover 442S can be substantially parallel to each other. In such configuration, the first mover 442F and the second mover 442S can have a side-by-side arrangement. Alternatively, the first mover 442F and the second mover 442S can have a top-bottom arrangement. In FIG. 4A, the first mover 442F and the second mover 442S have the top-bottom arrangement, such that the first mover 442F is positioned on top of or above the second mover 442S. Additionally, and/or in the alternative, the movers 442F, 442S, can have any other suitable positioning, such as end-to-end, for example.

Additionally, in FIG. 4A, the movers 442F, 442S, can be coupled to each other, such that as the first mover 442F moves in the first direction 445F or the second direction 445S, the second mover 442S also moves in the first direction 445F or the second direction 445S, which is opposite of the direction 445F, 445S, of the first mover 442F. For example, if the first mover 442F moves in the first direction 445F, then the second mover moves in the second direction 445S. Alternatively, if the first mover 442F moves in the second direction 445S, then the second mover 442S moves in the first direction 445F. In one embodiment, the movers 442F, 442S, can be coupled with a pinion 450. The pinion 450 can include a helical or a spur design, as non-exclusive examples. Alternatively, the pinion 450 may include any other suitable design or configuration. Additionally, and/or alternatively, the movers 442F, 442S, can be coupled to each other via any other suitable manner or method.

In various embodiments, the pinion 450 can engage the racks 449F, 449S, allowing the first mover 442F and second mover 442S to move relative to the rotational motion of the pinion 450. In some embodiments, the movers 442F, 442S, can move in a direction that is substantially parallel to the longitudinal axis 438 (illustrated as a dashed line). In other embodiments, the movers 442F, 442S, can move along the longitudinal axis 438.

In some embodiments, at least a portion of the movers 442F, 442S, can be at least partially positioned within the interior of the steering knob 440 and/or within the interior of the handle assembly 420. In other embodiments, the movers 442F, 442S, can be positioned solely within the interior of the handle assembly 420.

In certain embodiments, the pull wires 436F, 436S, can be coupled, secured or connected to the movers 442F, 442S. More specifically, in the embodiment illustrated in FIG. 4A, the first pull wire 436F can be coupled, secured or connected to the first mover 442F at or near an end of the first mover 442F that is adjacent to the steering knob 440. Alternatively, the first pull wire 436F can be coupled, secured or connected to the first mover 442F at any other suitable location or position. Furthermore, the second pull wire 436S is coupled, secured or connected to the second mover 442S at or near an end of the second mover 442S that is adjacent to the steering knob 440. Alternatively, the second pull wire 436S can be coupled, secured or connected to the second mover 442S at any other suitable location or position. The pull wires 436F, 436S, can be coupled, secured or connected to the movers 442F, 442S, in any suitable manner, i.e., via weld or solder joint, adhesive, or bonding material, as non-exclusive examples.

In certain embodiments, as the first mover 442F moves, the first pull wire 436F is moved. Similarly, as the second mover 442S moves, the second pull wire 436S is moved. For example, in FIG. 4A, the first mover 442F is engaged with the internal thread 444 of the steering knob 440 such that the internal thread 444 can act to push or move the first mover 442F in the first direction 445F or the second direction 445S. More specifically, the first mover 442F can include the knob engager 446 that engages or slots into the internal thread 444. In this embodiment, the knob engager 446 is positioned on the top surface of the first mover 442F. Accordingly, in the embodiment illustrated in FIG. 4A, when the steering knob 440 is manipulated, i.e., rotated about the longitudinal axis 438 in a clockwise or counter-clockwise direction, the rotational motion of the steering knob 440 and the internal thread 444 can cause movement of the first mover 442F in the first direction 445F or the second direction 445S relative to the steering knob 440, thereby translating the rotational motion of the steering knob 440 into linear movement of the first mover 442F. The linear movement of the first mover 442F can in turn cause the pinion 450 to rotate or move in the first direction 445F or the second direction 445S relative to the first mover 442F. The rotational motion of the pinion 450 can further cause the second mover 442S to move in the first direction 445F or the second direction 445S relative to the pinion 450, but in the direction 445F, 445S, that is opposite the first mover 442F, thereby translating the rotational motion of the pinion 450 into linear movement of the second mover 442S.

With this configuration, the pull wires 436F, 436S, which are coupled, secured or connected to the movers 442F, 442S, are moved. The movement of the first pull wire 436F and the second pull wire 436S functions to simultaneously push or loosen the first pull wire 436F while pulling or tightening the second pull wire 436S, and vice versa, which can articulate the balloon catheter 218 (illustrated in FIG. 2). In certain embodiments, articulate may also include providing bi-directional movement. In other words, as the operator rotates the steering knob 440, a portion of the balloon catheter 218 at or near the distal end 235 of the catheter shaft 232 (illustrated in FIG. 2) may then be articulated bi-directionally depending on the force being exerted by the first pull wire 436F and/or the second pull wire 436S.

FIG. 4B is a cross-sectional view of a portion of the embodiment of the handle assembly 420 and a portion of the embodiment of the steering assembly 426 taken on line 4B-4B in FIG. 4A. In this embodiment, both the steering knob 440 and the handle assembly 420 include or share the same longitudinal axis 438 (illustrated as a dashed line).

In the embodiment illustrated in FIG. 4B, the steering knob 440 includes the cylindrical cavity or shaft 447 that at least partially encircles or surrounds a portion of the handle assembly 420 and/or houses other components of the steering assembly 426. More specifically, the cylindrical cavity or shaft 447 at least partially houses the first drive member 442F, the second drive member 442S and the pinion 450 (illustrated in FIG. 4A), such that the first drive member 442F, the second drive member 442S and the pinion 450 can move in the first direction 445F (illustrated in FIG. 4A) and the second direction 445S (illustrated in FIG. 4A), while within the cylindrical cavity or shaft 447 of the steering knob 440.

Additionally, in this embodiment, the steering knob 440 includes the internal thread 444. The internal thread 444 is again positioned on the inner surface 448 of the steering knob 440, such that the knob engager 446 can engage the internal thread 444 of the steering knob 440. With this configuration, as the steering knob 440 is manipulated, i.e., rotated about the longitudinal axis 438 in a clockwise or counter-clockwise direction, the first mover 442F can move in the first direction 445F and the second mover 442S can move in the second direction 445S, or vice versa.

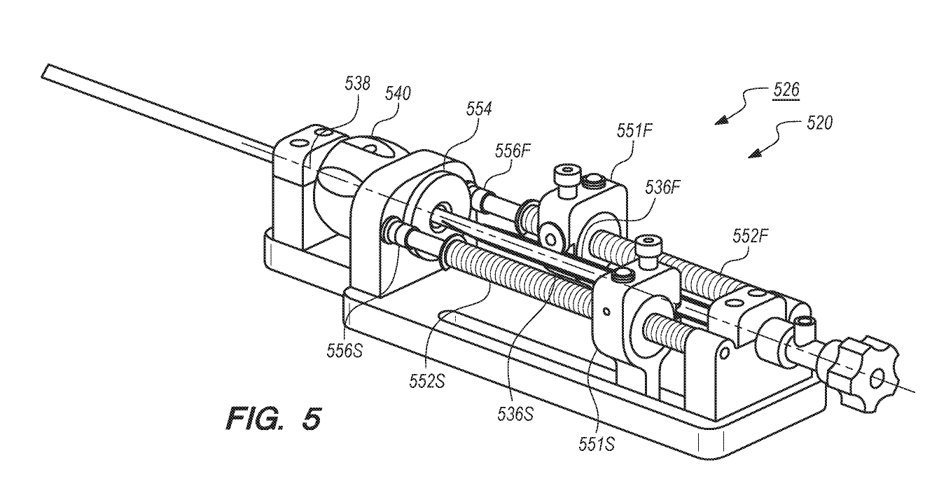

FIG. 5 is a perspective view of yet another embodiment of the steering assembly 526. In the embodiment illustrated in FIG. 5, the steering assembly 526 can include the first pull wire 536F, the second pull wire 536S, the steering knob 540, a first wire mover 551F, a second wire mover 551S, a first lead screw 552F and a second lead screw 552S. As referred to herein, the first wire mover 551F and the second wire mover 551S can be used interchangeably. Also, the first lead screw 552F and the second lead screw 552S may be used interchangeably.

In the embodiment illustrated in FIG. 5, the steering knob 540 is substantially cylindrical, i.e., similar to a knob, which includes the longitudinal axis 538 (illustrated as a dashed line). In this embodiment, the steering knob 540 is coupled to a gear 554, such that as the steering knob 540 is manipulated or rotated, the gear 554 can rotate in the same direction as the steering knob 540. The steering knob 540 can be coupled to the gear 554 in any suitable manner. In the embodiment illustrated in FIG. 5, the gear 554 includes a spur design. In alternative embodiments, the gear 554 can include any other suitable design.

The design of the first wire mover 551F and/or the second wire mover 551S can vary. In certain embodiments, the pull wires 536F, 536S, can be coupled, secured or connected to the wire movers 551F, 551S. More specifically, in FIG. 5, the first pull wire 536F is coupled, secured or connected to the first wire mover 551F, such that as the first wire mover 551F moves, the first pull wire 536F moves. The second pull wire 536S is also coupled, secured or connected to the second wire mover 551S, such that as the second wire mover 551S moves, the second pull wire 536S also moves. In the embodiment illustrated in FIG. 5, the pull wires 536F, 536S, are coupled, secured or connected to a top portion or surface of the wire movers 551F, 551S, respectively. Alternatively, the pull wires 536F, 536S, can be coupled, secured or connected to the wire movers 551F, 551S, at any other suitable location or position. Additionally, the pull wires 536F, 536S, can be coupled, secured or connected to the wire movers 551F, 551S, in any suitable manner, i.e., weld or solder joint, adhesive, bonding material or screw terminal, as non-exclusive examples.

The design of the first lead screw 552F and the second lead screw 552S can vary. In FIG. 5, the lead screws 552F, 552S, can include one of an external left-handed thread or an external right-handed thread. The lead screws 552F, 552S, can also rotate relative to the steering knob 540. In various embodiments, the lead screws 552F, 552S, can be coupled, secured or connected to the steering knob 540 so that rotation of the steering knob 540 causes the lead screws 552F, 552S, to rotate.

In the embodiment illustrated in FIG. 5, the first lead screw 552F is engaged with the first wire mover 551F and the second lead screw 552S is engaged with the second wire mover 551S. The lead screws 552F, 552S, can be engaged with the wire movers 551F, 551S, via any suitable manner or method. For example, in FIG. 5, the first lead screw 552F is engaged with an internal thread (not shown) of the first wire mover 551F and the second lead screw 552S is engaged with the internal thread (not shown) of the second wire mover 551S. In this embodiment, the first wire mover 551F includes the internal thread that matches or mates with the external thread of the first lead screw 552F and the second wire mover 551S includes the internal thread that matches or mates with the external thread of the second lead screw 552S. Accordingly, as the first lead screw 552F is rotated, the rotational motion of the first lead screw 552F can initiate movement of the first wire mover 551F, thereby translating the rotational motion of the first lead screw 552F into linear movement of the first wire mover 551F. Similarly, as the second lead screw 552S is rotated, the rotational motion of the second lead screw 552S can initiate movement of the second wire mover 551S, thereby translating the rotational motion of the second lead screw 552S into linear movement of the second wire mover 551S.

Additionally, in the embodiment illustrated in FIG. 5, the first lead screw 552F is coupled, secured or connected to a first lead screw gear 556F and the second lead screw 552S is coupled, secured and/or connected to a second lead screw gear 556S. The lead screws 552F, 552S, can be coupled, secured or connected to the lead screw gears 556F, 556S, in any suitable manner. In FIG. 5, the lead screw gears 556F, 556S, include a spur design. In alternative embodiments, the lead screw gears 556F, 556S, can include any other suitable design.

In certain embodiments, the lead screw gears 556F, 556S, can be engaged with the gear 554, such that as the steering knob 540 is manipulated or rotated, the gear 554 is rotated simultaneously in the same direction as the steering knob 540. Rotation of the gear 554 can thereby trigger the rotation of the lead screw gears 556F, 556S, and the lead screws 552F, 552S. Stated another way, the lead screw gears 556F, 556S, and the lead screws 552F, 552S, can rotate relative to the steering knob 540 and the gear 554. Additionally and/or alternatively, the lead screw gears 556F, 556S, can be engaged with the gear 554 via any other suitable manner or method.

Additionally, in the embodiment illustrated in FIG. 5, the first wire mover 551F and the second wire mover 551S can move in contrary and/or opposite directions. In this embodiment, the first pull wire 536F and the second pull wire 536S, which are connected to the first wire mover 551F and the second wire mover 551S, respectively, can be moved. The movement of the first pull wire 536F and second pull wire 536S functions to simultaneously push or loosen the first pull wire 536F while pulling or tightening the second pull wire 536S, and vice versa, which can articulate the balloon catheter 218 (illustrated in FIG. 2). In other words, as the operator rotates the steering knob 540, a portion of the balloon catheter 218 at or near the distal end 235 of the catheter shaft 232 (illustrated in FIG. 2) may then be articulated depending on the force being exerted by the first pull wire 536F and/or the second pull wire 536S.

It is appreciated that the embodiments of the steering assembly described in detail herein enable the realization of one or more certain advantages during the cryoablation procedure. With the various designs illustrated and described herein, the steering assembly can substantially reduce the likelihood of kinks, twists and/or fatigue of the pull wire(s). In other words, being able to simultaneously push or loosen and pull or tighten the pull wire(s) can function to substantially remove slack within the first pull wire when the second pull wire is in tension, and vice versa. Furthermore, the steering assembly can reduce the likelihood of steering backlash or hysteresis when the balloon catheter is manipulated to change directions during the cryoablation procedure.

It is understood that although a number of different embodiments of the steering assembly for the intravascular catheter system have been illustrated and described herein, one or more features of any one embodiment can be combined with one or more features of one or more of the other embodiments, provided that such combination satisfies the intent of the present invention.

While a number of exemplary aspects and embodiments of the steering assembly for the intravascular catheter system have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A steering assembly for an intravascular catheter system, the intravascular catheter system including a handle assembly and a balloon catheter having a catheter shaft that extends from the handle assembly, the catheter shaft having a distal end, the handle assembly including a longitudinal axis, the steering assembly comprising:
a first pull wire that extends between the handle assembly and the distal end of the catheter shaft;
a steering knob having an internal thread, the steering knob being rotatable about the longitudinal axis; and
a first mover that is connected to the first pull wire, the first mover including a knob engager that engages the internal thread of the steering knob so that rotation of the steering knob moves the first mover in a direction that is substantially parallel to the longitudinal axis, wherein movement of the first mover moves the first pull wire to articulate a portion of the balloon catheter near the distal end of the catheter shaft.

2. The steering assembly of claim 1 wherein the steering knob is coupled to the handle assembly.

3. The steering assembly of claim 2 wherein the steering knob encircles at least a portion of the handle assembly.

4. The steering assembly of claim 1 wherein the steering knob rotates relative to the first mover.

5. The steering assembly of claim 1 wherein the first mover moves along the longitudinal axis.

6. The steering assembly of claim 1 wherein movement of the first mover moves the first pull wire to articulate a portion of the balloon catheter near a distal end of a catheter sheath.

7. A steering assembly for an intravascular catheter system, the intravascular catheter system including a handle assembly and a balloon catheter having a catheter shaft that extends from the handle assembly, the catheter shaft having a distal end, the handle assembly including a longitudinal axis, the steering assembly comprising:
a first pull wire that extends between the handle assembly and the distal end of the catheter shaft;
a second pull wire that extends between the handle assembly and the distal end of the catheter shaft;
a steering knob having an internal thread, the steering knob being rotatable about the longitudinal axis;
a first mover that is connected to the first pull wire, the first mover including a knob engager that engages the internal thread of the steering knob so that rotation of the steering knob moves the first mover in a first direction that is substantially parallel to the longitudinal axis; and
a second mover that is connected to the second pull wire, the second mover being coupled to the first mover so that the second mover moves in a second direction that is substantially parallel to the longitudinal axis;
wherein movement of the first and second movers moves the first and second pull wires to articulate a portion of the balloon catheter near the distal end of the catheter shaft.

8. The steering assembly of claim 7 wherein the steering knob is coupled to the handle assembly.

9. The steering assembly of claim 8 wherein the steering knob encircles at least a portion of the handle assembly.

10. The steering assembly of claim 7 wherein the steering knob rotates relative to the first mover and the second mover.

11. The steering assembly of claim 7 wherein the first mover and the second mover are substantially parallel to each other.

12. The steering assembly of claim 11 wherein the first mover is positioned on top of the second mover.

13. The steering assembly of claim 7 wherein movement of the first and second movers moves the first and second pull wires to articulate a portion of the balloon catheter near a distal end of a catheter sheath.

14. The steering assembly of claim 7 further comprising a first rack that is connected to the first mover and a second rack that is connected to the second mover.

15. The steering assembly of claim 14 further comprising a pinion, wherein the pinion couples the first mover and the second mover to each other.

16. The steering assembly of claim 15 wherein the pinion includes one of a helical design and a spur design.

17. The steering assembly of claim 15 wherein the pinion engages both the first rack and the second rack.

18. The steering assembly of claim 17 wherein the pinion rotates relative to the first mover and the second mover.

19. The steering assembly of claim 7 wherein both the first mover and the second mover move along the longitudinal axis.

20. A steering assembly for an intravascular catheter system, the intravascular catheter system including a handle assembly and a balloon catheter having a catheter shaft that extends from the handle assembly, the catheter shaft having a distal end, the handle assembly including a longitudinal axis, the steering assembly comprising:
a first pull wire that extends between the handle assembly and the distal end of the catheter shaft;
a second pull wire that extends between the handle assembly and the distal end of the catheter shaft;
a steering knob having an internal thread, the steering knob being rotatable about the longitudinal axis;
a first mover that is connected to the first pull wire, the first mover having a first rack and a knob engager that engages the internal thread of the steering knob so that rotation of the steering knob moves the first mover in a first direction that is substantially parallel to the longitudinal axis;
a second mover that is connected to the second pull wire, the second mover including a second rack; and
a pinion that engages the first rack and the second rack so that the second mover moves in a second direction that is substantially parallel to the longitudinal axis;
wherein movement of the first and second movers moves the first and second pull wires to articulate a portion of the balloon catheter near the distal end of the catheter shaft.
